(19) United States
(12) Patent Application Publication
(10) Pub. No.: US 2009/0263343 A1
(43) Pub. Date: Oct. 22, 2009

(54) Deodorized cloth and method for deodorizing cloth

(76) Inventors: Midori Miyashita, Aichi (JP); Kohei Ohara, Kyoto (JP); Takuma Kawasaki, Ibaraki (JP); Shigeki Hayase, Kyoto (JP)

Correspondence Address: Kirschstein, Israel, Schiffmiller & Pieroni, P.C., 425 FIFTH AVENUE, 5TH FLOOR, NEW YORK, NY, 10016-2223, US

(21) Appl. No.: 12/310,899

(22) PCT Filed: Sep. 19, 2007
(86) PCT No.: PCT/JP2007/068153

§ 371 Date: May 7, 2009

(30) Foreign Application Priority Data
Sep. 25, 2006 (JP) 2006-258279
Mar. 12, 2007 (JP) 2007-062165

(51) Int. Cl. A61L 9/01

(52) U.S. Cl. 424/76.1

(57) ABSTRACT

Disclosed is a deodorized cloth which is produced by applying a deodorizing agent comprising active carbon and an amine compound in two layers on a cloth, thereby providing a durable deodorizing property against any type of odor to the cloth. Also disclosed is a method for deodorization processing of a cloth. A resin composition comprising active carbon is applied as the first layer, and a resin composition comprising a deodorizing agent comprising an amine compound is applied as the second layer. In the resin composition for use as the first layer, a mixture of a deodorizing agent comprising a metal compound and a flame retardant agent may be used. Preferably, the resin composition for use as the second layer is applied in a dot-like pattern.

### TECHNICAL FIELD

The present invention relates to a deodorized cloth having effectively a durable deodorizing property and a method for preparation of it.

### BACKGROUND TECHNOLOGY

Recently with increasing pet lovers and anti-smoking people, it has been strongly required to deodorize dog odor and cigarette odor in an automotive interior material or sheet.

As deodorants, there have been used, for example, as chemical adsorbents calcium carbonate being a basic material for an acid gas, and aluminum sulfate being an acid material for basic gas, and as physical adsorbents active carbon and diatomaceous earth. However, though it has been known that the main odor of pets, particularly dogs, is higher fatty acid and the aldehyde (isovaleric aldehyde etc.), a liquid deodorant for those aldehydes has not be found. Even though solid powders, such as copper oxide, chitosan, manganese dioxide, catechin, cyclodextrin and the like are used, effective deodorizing property can not be obtained.

With use of active carbon, effective deodorant property can be obtained, but the odor deodorized is limited and accordingly it is required to provide a method for deodorant treatment in which deodorant effects over a wide range can be obtained.

For example, Patent Documents 1-5 disclose various deodorant compositions, but they have the following problems and deodorizing effects over a wide range can not be expected.

1) By carelessly mixing various deodorants to obtain a deodorant having widely deodorant effects, the property as the deodorant is lowered by the reaction between the deodorants so that the original effects can not be obtained.
2) They are not expected to have a high effect for the problem living malodors, aldehydes, such as acetaldehyde being a component in cigarette odor, isovaleric aldehyde being a component in pet odor, and nonenal being the main component of aging odor (human body odor associated with aging).
3) Hydrazide compounds being an amine compound are useful for odor-reducing of aldehydes, but with use of a mixture of active carbon, porous silicon dioxide or zinc oxide, the odor-reducing property for aldehyde is lowered as time passed.

### Patent Document 1: JP 2005-198684 A

### Patent Document 2: JP 2001-218668 A

### Patent Document 3: JP 2000-014520 A

### Patent Document 4: JP 08-280781 A

### Patent Document 5: JP 3765147 B

### DISCLOSURE OF THE INVENTION

### Problems to be Solved by the Invention

An object of the present invention is to provide a deodorized cloth having effectively a durable deodorizing property over a wide range and an efficient method for preparation of it.

### Means for Solving the Problems

In the present invention, the above problems have been dissolved by coating an active carbon and a deodorizing agent comprising an amine compound as overlapping double layers on a cloth.

Namely, a deodorized cloth is prepared by coating the first deodorant layer consisting of a resin composition comprising an active carbon on a surface of a cloth and then the second deodorant layer consisting of a resin composition comprising a deodorant of amine compounds to obtain the desired effects.

In the preparation of the deodorized cloth, in which a resin composition comprising an active carbon is coated on a cloth to form a first deodorant layer and then a resin composition comprising a deodorant consisting of an amine compounds is applied to form a second deodorant layer, the function of both of the active carbon and the deodorant consisting of amine compounds is effectively fulfilled. To the first deodorant layer formed by the resin composition comprising the active carbon, a deodorant consisting of a metal compound may be added.

The resin composition (the second deodorant layer) comprising a deodorant consisting of amine compounds is preferably applied to form a pattern having a space. The formation of a pattern having the space means that the resin composition is not coated as a layer covering the whole surface, but is coated to form intermittently uncoated portions. For example the coating is preferred to have dot-like patterns. The dot-like patterns are those having repeatedly arranged figures such as dots, lines, circles, polygons and the like. As the coating methods, any of screen printing, transfer printing, inkjet printing, gravure roll coating and like is applicable. By forming the second deodorant layer to have a pattern such as dot-like patterns, there can be obtained products superior in feeling, antistatic property and the like.

As deodorant amine compounds, there are preferably used compounds having a primary amine group in the molecule, such as hydrazine compounds effective to adsorb formaldehyde, acetaldehyde, acetic acid and the like. As the hydrazine compounds, adipic acid dihydrazide, azelaic acid dihydrazide, succinic acid dihydrazide, glutaric acid dihydrazide, oxalic acid dihydrazide, suberic acid dihydrazide, sebacic acid dihydrazide, dodecanoic acid dihydrazide, pimelic acid dihydrazide, malonic acid dihydrazide, isophthalic acid dihydrazide, terephthalic acid dihydrazide, polyacrylic acid dihydrazide and the like are exemplified.

Further, among the metal compounds functioning as a deodorant, there are exemplified metal compounds containing zinc or copper which demonstrates deodorant effect to odors of hydrogen sulfide and mercaptans, such as zinc or copper oxide, hydroxide, chloride, sulfate, acetate, citrate and the like. Particularly zinc silicate, zinc oxide and the like are preferably used.

Additionally, in the present invention, by using a flame retardant mixed in the resin composition comprising an active carbon (the first deodorant layer), there may be obtained a product superior in both of flame retardant and deodorant effects. As the flame retardants, for example bromine compounds used in automotive interior sheets, a mixture of them with antimony compounds, phosphoric acid compounds (e.g. phosphoric ester compounds, condensed Phosphoric ester compounds, phosphoric amide compounds, phosphoric melamine compounds and the like), hydroxide metal compounds and the like may be effectively used.

In the present invention, with separately coating the active carbon functioning as physical adsorbent and the deodorant consisting of amine compound to form double layers, each of them shows efficiently the deodorant function. Further, when a flame retardant is used, by adding it in a layer different from amine compound can be obtained a product superior in both of flame retardant and deodorant effects.

As the binders used in the resin composition of the present invention, general binders for textile treatment or processing may be used. Acrylic resins and urethane resins are preferably used.

Further, in the present invention, the resin composition containing an active carbon (the first deodorant layer) may be coated in an amount of 20-200 g/m², preferably 40-150 g/m² on dry basis. The resin composition containing amine compounds (the second deodorant layer) is preferably coated in an amount of 8-100 g/m² on dry basis.

The dry weight ratio of resin and active carbon in the first deodorant layer is preferably about 1:2-5:1, and particularly about 1:1-3:1. When a flame retardant is used in the first deodorant layer, the preferable dry weight ratio of the resin and the flame retardant is about 2:1-1:3. The dry weight ratio of the resin and amine compound used in the second deodorant layer is preferably about 1:4-3:1, and particularly about 1:3-2:1.

Further, when a deodorant agent comprising a metal compound is mixed in the first deodorant layer, the used amount may be the same as of the active carbon or less on dry basis.

### EFFECT OF THE INVENTION

In the present invention, a product having comprehensively a deodorant effect (a Product effective in deodorizing both of pet odor and cigarette odor) can be produced by applying simple coating method to a cloth. For example, the treated cloth of the present invention shows stably deodorant effects for 10 days or more in acetaldehyde gas recharging test, and the total adsorption amount is very superior in 3900 mg/m² or more.

### BEST MODE FOR EXECUTING THE INVENTION

Nest, the present invention is described in detail according to Examples. The tests carried out in Examples are as follows:

### <Deodorant Test>

A cloth having a length of 8 cm and a width of 10 cm is put in a scent bag, a prepared gas is sealed in the bag, and the bag is stood in a room at 20%. The residual gas content after a given time is measured by a gas detector tubes manufactured by Gastec Corporation. The numeral values in Tables are deodorization rate % shown in the following equation.

Deodorization Rate=[(gas content about gas alone after a given time−gas content about a treated cloth after a given time)/gas content about gas alone after a given time]×100

### <Flame Retardant Test>

According to EMVSS No. 302 (JIS D1201 horizontal method)

### <Antistatic Test>

With use of Super Insulation Resistance Tester manufactured by Kasuga Electric Works Ltd., a surface resistivity of the test cloth is measured. The applied voltage is 90 V.

### Example 1

Two coating compositions shown in Table 1 as the first layer and the second layer were prepared, the first coating composition was coated on a polyester fabric (400 g/m²) with a knife coater, dried at 150° C. for 2 minutes, and then the second coating composition was applied by a screen printing method (opening rate of the screen mesh: 50%) and dried at 150° C. for 2 minutes.

TABLE 1

| | 1st layer | 2nd layer |
|---|---|---|
| Coating composition (wt. parts) | | |
| acrylic resin binder (solid 50%) | 35 | 30 |
| <flame retardant> | | |
| antimonic bromide type flame | 30 | — |
| retardant (solid 70%) | | |
| <deodrizing agent> | | |
| aliphatic amine compound | — | 30 |
| zinc oxide compound | 3 | — |
| active carbon (solid 50%) | 20 | — |
| thickener (hydroxyethylcellulose) | 0.6 | 0.6 |
| water | 6 | 20 |
| Total amount | 94.6 | 80.6 |
| Dry coated amount (g/m²) | 88 | 35 |

The product has good feeling and the value of the antistatic property is excellent in 10⁵Ω or less. Further, in the flame retardant test, a good result is obtained as self-extinction (evaluation: 0)

The test results of deodorant test are as shown in Table 3A-3B and Table 4. Namely, in the acetaldehyde gas recharging test, the deodorant effect is stably maintained for more than 10 days, and the total adsorption amount is very superior in more than 3900 mg/m² (See Tables 3A-3B). Further, in the deodorant tests for formaldehyde, acetic acid, ammonia, trimethylamine, hydrogen sulfide, methyl mercaptan and toluene, the deodorization rates after 4 hours as shown in Table are obtained. It is recognized that stable deodorant effects are widely obtained.

### [Control 1]

The coating composition shown in Table 2 was coated on same polyester fabric as in Example 1 by knife coating method, and then dried for 2 minutes at 150° C.

TABLE 2

| | monolayer |
|---|---|
| | coating |
| Coating composition (wt. parts) | |
| acrylic resin binder (solid 50%) | 40 |
| <flame retardant> | |
| antimonic bromide type flame | 30 |
| retardant (solid 70%) | |
| <deodrizing agent> | |
| aliphatic amine compound | 5 |
| zinc oxide compound | 3 |
| active carbon (solid 50%) | 20 |
| thickener (hydroxyethylcellulose) | 0.2 |
| water | 11.8 |
| Total amount | 110 |
| Dry coated amount (g/m²) | 100 |

The antistatic value of this product is less than 10⁵Ω which is good value similar to Example 1. However, the result of deodorant test is bad as shown in Table 5, and in acetaldehyde gas recharging test, the deodorant effect disappears after about 3 days. The total adsorption amount is 688 mg/m² which is not good.

TABLE 3A
Acetaldehyde

| | | |
|---|---|---|
| | deodori- | total |
| gas conc. (ppm) | zation | adsorption |

| Time | | treated | rate | amount |
|---|---|---|---|---|
| (hrs.) | gas only | cloth | (%) | (mg/m²) |
| 0 | 750 | 750 | 0.0 | 0 |
| 7 | 625 | 60 | 90.4 | 492 |
| gas recharging test | | | | |
| 7.5 | 750 | 750 | 0.0 | 492 |
| 23.5 | 625 | 50 | 92.0 | 992 |
| 24 | 750 | 750 | 0.0 | 992 |
| 31 | 687.5 | 220 | 68.0 | 1399 |
| 31.5 | 750 | 750 | 0.0 | 1399 |
| 47.5 | 625 | 190 | 69.6 | 1778 |
| 48 | 750 | 750 | 0.0 | 1778 |
| 55 | 625 | 450 | 28.0 | 1930 |
| 55.5 | 750 | 750 | 0.0 | 1930 |
| 71.5 | 750 | 437.5 | 41.7 | 2202 |
| 72 | 750 | 750 | 0.0 | 2202 |
| 79 | 600 | 500 | 16.7 | 2290 |
| 79.5 | 750 | 750 | 0.0 | 2290 |
| 95.5 | 650 | 475 | 26.9 | 2442 |
| 96 | 750 | 750 | 0.0 | 2442 |
| 103 | 625 | 600 | 4.0 | 2464 |
| 103 | 750 | 750 | 0.0 | 2464 |
| 119 | 700 | 350 | 50.0 | 2768 |

TABLE 3B
Acetaldehyde

| | | |
|---|---|---|
| | deodori- | total |
| gas conc. (ppm) | zation | adsorption |

| Time | | treated | rate | amount |
|---|---|---|---|---|
| (hrs.) | gas only | cloth | (%) | (mg/m²) |
| 119.5 | 750 | 750 | 0.0 | 2768 |
| 126.5 | 625 | 500 | 20.0 | 2877 |
| 127 | 750 | 750 | 0.0 | 2877 |
| 143 | 675 | 525 | 22.2 | 3008 |
| 143.5 | 750 | 750 | 0.0 | 3008 |
| 150.5 | 625 | 600 | 4.0 | 3029 |
| 151 | 750 | 750 | 0.0 | 3029 |
| 167 | 700 | 625 | 10.7 | 3095 |
| 167.5 | 750 | 750 | 0.0 | 3095 |
| 174.5 | 700 | 700 | 0.0 | 3095 |
| 174.5 | 750 | 750 | 0.0 | 3095 |
| 190.5 | 625 | 450 | 28.0 | 3247 |
| 191 | 750 | 750 | 0.0 | 3247 |
| 198 | 750 | 675 | 10.0 | 3312 |
| 198 | 750 | 750 | 0.0 | 3312 |
| 214 | 675 | 500 | 25.9 | 3465 |
| 214.5 | 750 | 750 | 0.0 | 3265 |
| 221.5 | 725 | 700 | 3.4 | 3486 |
| 221.5 | 750 | 750 | 0.0 | 3486 |
| 237.5 | 700 | 500 | 28.6 | 3661 |
| 238 | 750 | 750 | 0.0 | 3661 |
| 245 | 687.5 | 625 | 9.1 | 3715 |
| 245.5 | 750 | 750 | 0.0 | 3715 |
| 261.5 | 700 | 600 | 14.3 | 3802 |
| 262 | 750 | 750 | 0.0 | 3802 |
| 269 | 750 | 750 | 0.0 | 3802 |
| 269 | 750 | 750 | 0.0 | 3802 |
| 285 | 625 | 500 | 20.0 | 3911 |
| 285.5 | 750 | 750 | 0.0 | 3911 |
| 292.5 | 700 | 700 | 0.0 | 3911 |
| 293 | 750 | 750 | 0.0 | 3911 |
| 309 | 675 | 675 | 0.0 | 3911 |

TABLE 4

deodorization rate %

| | | untreated |
|---|---|---|
| | Example 1 | cloth |
| formaldehyde | 99 | 60 |
| acetic acid | 91 | 75 |
| ammonia | 99 | 65 |
| trimethylamine | 91 | 25 |
| hydrogen sulfide | 100 | 15 |
| methyl mercaptan | 94 | 7 |
| toluene | 98 | 32 |

TABLE 5
Acetaldehyde

| | | |
|---|---|---|
| | deodori- | total |
| gas content (ppm) | zation | adsorption |

| Time | | treated | rate | amount |
|---|---|---|---|---|
| (hrs.) | gas only | cloth | (%) | (mg/m²) |
| 0 | 750 | 750 | 0.0 | 0 |
| 7 | 650 | 210 | 67.7 | 383 |
| gas recharging test | | | | |
| 7.5 | 750 | 750 | 0.0 | 383 |
| 23.5 | 650 | 500 | 23.1 | 514 |
| 24 | 750 | 750 | 0.0 | 514 |
| 31 | 700 | 675 | 3.6 | 535 |
| 31 | 750 | 750 | 0.0 | 535 |
| 47.5 | 600 | 475 | 20.8 | 644 |
| 48 | 750 | 750 | 0.0 | 644 |
| 55 | 625 | 625 | 0.0 | 644 |
| 55.5 | 750 | 750 | 0.0 | 644 |
| 72 | 625 | 575 | 8.0 | 688 |

### Example 2

Two coating compositions (the first layer and the second layer) as shown in Table 6 were prepared. On a polyester fabric (weight of 400 g/m²) the first coating composition was applied with a knife coater, dried for 2 minutes at 150° C., then the second coating composition was applied with a screen printing method (opening rate of the screen mesh: 50%) and dried for 2 minutes at 150° C.

TABLE 6

| | 1st layer | 2nd layer |
|---|---|---|
| Coating composition (wt. parts) | | |
| acrylic resin binder (solid 50%) | 15 | 10 |
| <flame retardant> | — | — |
| <deodrizing agent> | | |
| aliphatic amine compound | — | 5 |
| zeolite carried type amine | — | 5 |
| compound | | |
| zinc oxide compound | 5 | — |
| active carbon (solid 50%) | 10 | — |
| thickener (hydroxyethylcellulose) | 0.2 | 0.2 |
| water | 22 | 17 |
| Total amount | 52.2 | 37.2 |
| dry coated amount (g/m²) | 49.2 | 37 |

The product is superior in feeling and has a good antistatic value of less than 10⁵Ω. Further, the result of deodorant test is excellent, and as shown in Table 7, in acetaldehyde gas recharging test, stable deodorant effects are recognized and the total adsorption amount after 6 days is 3600 mg/m² which is excellent.

TABLE 7
Acetaldehyde

| | | |
|---|---|---|
| | deodori- | total |
| gas conc. (ppm) | zation | adsorption |

| Time | | treated | rate | amount |
|---|---|---|---|---|
| (hrs.) | gas only | cloth | (%) | (mg/m²) |
| 0 | 750 | 750 | 0.0 | 0 |
| 7 | 650 | 210 | 99.7 | 564 |
| gas recharging test | | | | |
| 7.5 | 750 | 750 | 0.0 | 564 |
| 23.5 | 625 | 4 | 99.4 | 1105 |
| 24 | 750 | 750 | 0.0 | 1105 |
| 31 | 700 | 60 | 91.4 | 1662 |
| 31.5 | 750 | 750 | 0.0 | 1662 |
| 47.5 | 650 | 100 | 84.6 | 2141 |
| 48 | 750 | 750 | 0.0 | 2141 |
| 55 | 700 | 375 | 46.4 | 2424 |
| 55.5 | 750 | 750 | 0.0 | 2424 |
| 71.5 | 650 | 450 | 30.8 | 2598 |
| 72 | 750 | 750 | 0.0 | 2598 |
| 79 | 750 | 600 | 20.0 | 2728 |
| 79 | 750 | 750 | 0.0 | 2728 |
| 95 | 625 | 230 | 63.2 | 3072 |
| 95.5 | 750 | 750 | 0.0 | 3072 |
| 102.5 | 725 | 600 | 17.2 | 3181 |
| 102.5 | 750 | 750 | 0.0 | 3181 |
| 118.5 | 625 | 450 | 28.0 | 3333 |
| 119 | 750 | 750 | 0.0 | 3333 |
| 126 | 625 | 500 | 20.0 | 3442 |
| 126 | 750 | 750 | 0.0 | 3442 |
| 142 | 700 | 550 | 21.4 | 3573 |
| 142.5 | 750 | 750 | 0.0 | 3573 |
| 149.5 | 750 | 625 | 16.7 | 3681 |

### Examples 3 and 4

In each Example, two coating compositions (the first layer and the second layer) as shown in Table 8 were prepared, the first coating composition was coated on a whole surface of a polyester rising tricot (weight of 400 g/m²) by a knife coater, and dried for 2 minutes at 150° C. Then the second coating composition was coated similarly on the whole surface by a knife coater, and dried for 2 minutes at 150° C.

TABLE 8

| | Example 3 | | Example 4 | |
|---|---|---|---|---|
| | 1st layer | 2nd layer | 1st layer | 2nd layer |
| Coating composition (wt. parts) | | | | |
| acrylic resin binder (solid 50%) | 35 | 30 | 35 | 30 |
| <flame retardant> | | | | |
| antimonic bromide type flame | 30 | — | 30 | — |
| retardant (solid 70%) | | | | |
| <deodrizing agent> | | | | |
| aliphatic amine compound | — | 30 | — | 30 |
| zinc oxide compound | 3 | — | 3 | — |
| active carbon (solid 50%) | 20 | — | 20 | — |
| thickener (hydroxyethylcellulose) | 0.6 | 0.6 | 0.6 | 0.6 |
| water | 6 | 20 | 6 | 6.7 |
| Total amount | 94.6 | 80.6 | 94.6 | 47.3 |
| dry coated amount (g/m²) | 88 | 20 | 87 | 22 |

The products obtained in Examples 3 and 4, in comparison with those obtained in Examples 1 and 2, become somewhat hard but the antistatic property is less than 10⁵Ω which is excellent.

Further, as shown in Table 9, the result of deodorant test is also excellent, and in acetaldehyde gas recharging test, stable deodorant effects for 7 days or more are recognized and the total adsorption amount after 7 days is more than 1480 mg/m² in Example 3 and more than 958 mg/m² in Example 4, each of which is excellent.

TABLE 9
Acetaldehyde

| | gas only | Treated cloth in Example 3 | | | Treated cloth in Example 4 | | |
|---|---|---|---|---|---|---|---|
| | gas | gas | deodori- | total | gas | deodori- | total |
| | concen- | concen- | zation | adsorbed | concen- | zation | adsorbed |
| Time | tration | tration | rate | amount | tration | rate | amount |
| (hrs.) | (ppm) | (ppm) | (%) | (mg/m²) | (ppm) | (%) | (mg/m²) |
| 0 | 750 | 750 | 0.0 | 0 | 750 | 0.0 | 0 |
| 7 | 625 | 225 | 64.0 | 348 | 250 | 60.0 | 326 |
| gas recharging test | | | | | | | |
| 7.5 | 750 | 750 | 0.0 | 348 | 750 | 0.0 | 326 |
| 23.5 | 625 | 250 | 60.0 | 675 | 425 | 32.0 | 501 |
| 24 | 750 | 750 | 0.0 | 675 | 750 | 0.0 | 501 |
| 31 | 675 | 500 | 25.9 | 827 | 600 | 11.1 | 566 |
| 31.5 | 750 | 750 | 0.0 | 827 | 750 | 0.0 | 566 |
| 47.5 | 750 | 450 | 40.0 | 1088 | 575 | 23.3 | 718 |
| 48 | 750 | 750 | 0.0 | 1088 | 750 | 0.0 | 718 |
| 55 | 650 | 575 | 11.5 | 1153 | 600 | 7.7 | 762 |
| 55.5 | 750 | 750 | 0.0 | 1153 | 750 | 0.0 | 762 |
| 71.5 | 700 | 625 | 10.7 | 1219 | 625 | 10.7 | 827 |
| 72 | 750 | 750 | 0.0 | 1219 | 750 | 0.0 | 827 |
| 79 | 625 | 600 | 4.0 | 1241 | 600 | 4.0 | 849 |
| 79 | 750 | 750 | 0.0 | 1241 | 750 | 0.0 | 849 |
| 95 | 525 | 400 | 23.8 | 1349 | 450 | 14.3 | 914 |
| 95.5 | 750 | 750 | 0.0 | 1349 | 750 | 0.0 | 914 |
| 102.5 | 675 | 575 | 14.8 | 1436 | 625 | 7.4 | 958 |
| 102.5 | 750 | 750 | 0.0 | 1436 | 750 | 0.0 | 958 |
| 118.5 | 675 | 625 | 7.4 | 1480 | 675 | 0.0 | 958 |

1-8. (canceled)

9. A deodorized cloth, comprising: first and second deodorant layers on a surface of the cloth, the first deodorant layer consisting of a resin composition comprising an active carbon, and the second deodorant layer consisting of a resin composition comprising a deodorant agent of an amine compound.

10. The deodorized cloth of claim 9, wherein the first deodorant layer includes a flame retardant.

11. The deodorized cloth of claim 9, wherein a deodorant consisting of a metal compound is added to the first deodorant layer.

12. The deodorized cloth of claim 9, wherein the second deodorant layer is formed as a dot-like pattern.

13. A method of applying a deodorizing property to a cloth, comprising the steps of: coating a resin composition containing active carbon on a surface of the cloth to form a first deodorant layer; and then coating a resin composition containing a deodorant of amine compounds to form a second deodorant layer.

14. The method of claim 13, in which a flame retardant is comprised in the resin composition comprising the active carbon.

15. The method of claim 13, adding a deodorant consisting of a metal component to the resin composition comprising the active carbon.

16. The method of claim 13, coating the resin composition comprising the deodorant of amine compounds as a dot-like pattern.
